(19) United States
(12) Patent Application Publication
(10) Pub. No.: US 2011/0190692 A1
(43) Pub. Date: Aug. 4, 2011

(54) VASODILATOR DELIVERY REGULATED BY BLOOD PRESSURE OR BLOOD FLOW

(75) Inventor: Venkatesh Manda, Stillwater, MN (US)

(73) Assignee: Medtronic, Inc., Minneapolis, MN (US)

(21) Appl. No.: 12/696,922

(22) Filed: Jan. 29, 2010

(51) Int. Cl. A61M 5/172; G08B 21/00

(52) U.S. Cl. 604/66; 604/505; 340/540

(57) ABSTRACT

The effectiveness of a vasodilator delivered to a patient and/or the operation of the fluid delivery device from which the vasodilator is delivered are evaluated based on feedback from one or more sensors implanted within the patient. A fluid delivery system includes a fluid delivery device, a sensor, and a processor. The fluid delivery device is configured to deliver a vasodilator. The sensor is configured to sense at least one of blood pressure or blood flow in one of a ventricle or an atria of a heart, a pulmonary artery, and a renal vessel. The processor is configured to trigger a therapeutic action when the sensed at least one of blood pressure or blood flow traverses the threshold.

### TECHNICAL FIELD

This disclosure relates generally to implantable medical devices and, more particularly, to implantable fluid delivery systems.

### BACKGROUND

A variety of medical devices are used for chronic, i.e., long-term, delivery of fluid therapy to patients suffering from a variety of conditions, such as chronic pain, tremor, Parkinson's disease, epilepsy, urinary or fecal incontinence, sexual dysfunction, obesity, spasticity, or gastroparesis. For example, pumps or other fluid delivery devices can be used for chronic delivery of therapeutic agents, such as drugs to patients. These devices are intended to provide a patient with a therapeutic output to alleviate or assist with a variety of conditions. Such devices may be implanted in a patient and provide a therapeutic output under specified conditions on a recurring basis.

One type of implantable fluid delivery device is a drug infusion device that can deliver a fluid medication to a patient at a selected site. A drug infusion device may be implanted at a location in the body of a patient and deliver a fluid medication through a catheter to a selected delivery site in the body. Drug infusion devices, such as implantable drug pumps, commonly include a reservoir for holding a supply of the therapeutic substance, such as a drug, for delivery to a site in the patient. The fluid reservoir can be self-sealing and percutaneously accessible through one or more ports. A pump may be fluidly coupled to the reservoir for delivering the therapeutic substance to the patient. A catheter may provide a pathway for delivering the therapeutic substance from the pump to the delivery site in the patient.

### SUMMARY

In general, this disclosure describes techniques for evaluating the effectiveness of treating a patient with a vasodilator and/or the operation of a fluid delivery device by which the vasodilator is delivered.

In one example, a fluid delivery system includes a fluid delivery device, a sensor, and a processor. The fluid delivery device is configured to deliver a vasodilator. The sensor is configured to sense at least one of blood pressure or blood flow in one of a ventricle or an atria of a heart, a pulmonary artery, and a renal vessel. The processor is configured to trigger a therapeutic action when the sensed at least one of blood pressure or blood flow traverses the threshold.

In another example, a fluid delivery system includes a primary fluid delivery apparatus, a reserve fluid delivery apparatus, a sensor, and a processor. The primary fluid delivery apparatus and the reserve fluid delivery apparatus are configured to deliver a vasodilator. The sensor is configured to sense at least one of blood pressure or blood flow in one of a ventricle or an atria of a heart, a pulmonary artery, and a renal vessel. The processor is configured to switch delivery of the vasodilator from the primary delivery apparatus to the reserve fluid delivery apparatus when the sensed at least one of blood pressure or blood flow traverses the threshold.

In another example, a method includes delivering a vasodilator with a fluid delivery device, sensing at least one of blood pressure or blood flow in one of a ventricle or an atria of a heart, a pulmonary artery, and a renal vessel with a sensor, and triggering a therapeutic action by the fluid delivery device when the sensed at least one of blood pressure or blood flow traverses the threshold.

In another example, a fluid delivery system includes means for delivering a vasodilator, means for sensing at least one of blood pressure or blood flow in one of a ventricle or an atria of a heart, a pulmonary artery, and a renal vessel, and means for triggering a therapeutic action when the sensed at least one of the sensed blood pressure or blood flow traverses the threshold.

The details of one or more examples disclosed herein are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example of a fluid delivery system including an implantable fluid delivery device configured to deliver a therapeutic agent to a patient via a catheter.

FIG. 2 is functional block diagram illustrating an example of the implantable fluid delivery device of FIG. 1.

FIG. 3 is a functional block diagram illustrating an example of an external programmer for the system of of FIG. 1.

FIG. 4 is a flow chart illustrating an example method of triggering therapeutic actions in response to patient blood pressure readings.

### DETAILED DESCRIPTION

Medical devices are useful for treating, managing or otherwise controlling various patient conditions or disorders including, e.g., pain (e.g., chronic pain, post-operative pain or peripheral and localized pain), tremor, movement disorders (e.g., Parkinson's disease), diabetes, epilepsy, neuralgia, chronic migraines, urinary or fecal incontinence, sexual dysfunction, obesity, gastroparesis, mood disorders, or other disorders. Some medical devices, referred to herein generally as fluid delivery devices may be configured to deliver one or more therapeutic fluids, alone or in combination with other therapies, such as electrical stimulation, to one or more target sites within a patient. For example, in some cases, a fluid delivery device may deliver pain-relieving drug(s) to patients with chronic pain, insulin to a patient with diabetes, or other fluids to patients with different disorders. The device may be implanted in the patient for chronic therapy delivery (i.e., longer than a temporary, trial basis) or temporary delivery.

The operation of fluid delivery devices may be defined by a number of parameters related to the amount and timing of therapeutic fluid delivery to a patient. In some examples, the therapeutic fluid delivery parameters are defined in a dosing or therapy program and/or therapy schedule. A dosing or therapy program generally may refer to a program sent to an implantable fluid delivery device by a programming device to cause the fluid delivery device to deliver fluid at a certain rate and at a certain time. The dosing program may include, for example, definitions of a priming bolus, a bridging bolus, a supplemental bolus, and a therapy schedule. A dosing program may include additional information, such as patient information, permissions for a user to add a supplemental bolus, as well as limits on the frequency or number of such boluses, historical therapy schedules, fluid or drug information, or other information.

A therapy schedule generally refers to a rate (which may be zero) at which to administer one or more therapeutic fluids at specific times to a patient. In particular, the therapy schedule may define one or more programmed doses, which may be periodic or aperiodic including, e.g., a rate of fluid delivery and different times and/or time durations for which to deliver the dose. Dose generally refers to the amount of therapeutic fluid delivered over a period of time, and may change over the course of a therapy schedule such that a fluid may be delivered at different rates at different times.

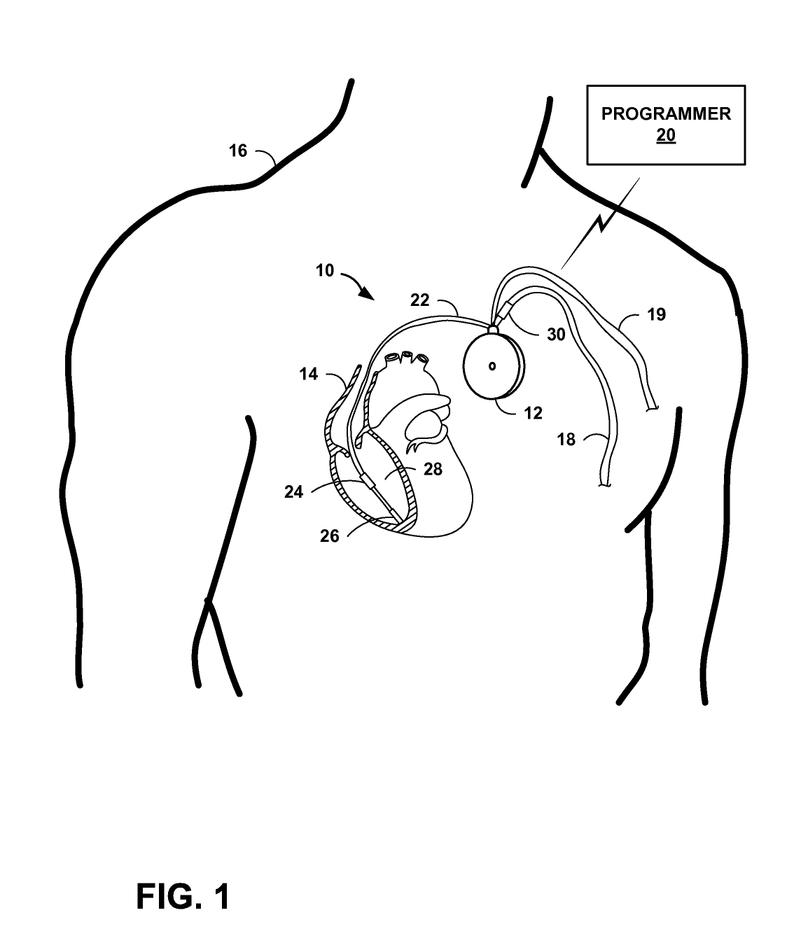

FIG. 1 is a conceptual diagram illustrating an example of a therapy system 10, which includes implantable medical device (IMD) 12, catheters 18 and 19, external programmer 20, and lead 22. IMD 12 is connected to catheters 18 and 19 to deliver at least one therapeutic agent, such as a pharmaceutical agent, pain relieving agent, anti-inflammatory agent, gene therapy agent, or the like, to a target site within patient 16. Example therapeutic agents that IMD 12 can be configured to deliver include vasodilators, which may include renal enhancing proteins and peptides. IMD 12 is also connected to lead 22, which includes sensor 24 and electrode 26 arranged toward a distal end of the lead. In the example of FIG. 1, sensor 24 and electrode 26 are positioned within right ventricle 28 of heart 14. In other examples, system 10 may include one or more sensors arranged in other locations within patient 16 including, e.g., the left ventricle, an atria, the pulmonary artery (PA) or a renal vessel of the patient. As described in detail below, IMD 12 is configured to measure at least one of the blood pressure or blood flow of patient 16 via sensor 24 and electrical activity of heart 14 via electrode 26. Electrode 26 may be employed as a pair of lead electrodes configured for bipolar sensing or in combination with an electrode connected to or a part of the housing of IMD 12 for unipolar sensing of the electrical activity of heart 14.

In the following examples, IMD 12 is configured to deliver a vasodilator to one or more target sites within patient 16 to treat conditions including, e.g., hypertension, heart failure, kidney failure, and/or angina. Vasodilators relax the smooth muscle in blood vessels, which reduces the pressure in the vessels by causing them to dilate. Techniques described in this disclosure may be directed to automatically evaluating the effectiveness of treating patient 16 with a therapeutic fluid such as a vasodilator and/or the operation of IMD 12 to deliver the therapeutic fluid to the patient. In some examples, IMD 12 may be configured to deliver one or more vasodilators including, e.g., an angiotensin-converting enzyme (ACE) inhibitor, an angeotensin receptoblocker (ARB), or a prostacyclin. Vasodilators employed in the disclosed examples may have other therapeutic properties including, e.g., enhancing renal system function. Example vasodilators deliverable by IMD 12 and including renal enhancing proteins or peptides include atrial natriuretic peptides (ANP), vessel-dilator, and kaliuretics.

The disclosed examples include a sensor implanted and configured to sense at least one of blood pressure or blood flow within patient 16. The sensor may, in some examples, include a pressure sensor configured to measure blood pressure directly and/or the pressure measurements of which may be used to extrapolate blood flow. In other examples, blood flow within patient 16 may be measured by an optical blood oxygen saturation sensor configured to measure changes in blood oxygen levels over a period of time to determine blood flow. The implanted sensor may be employed as a measurement of the effectiveness of the treatment of patient 16 with the vasodilator and/or the operation of IMD 12. In the event the pressure sensor senses that the blood pressure has exceeded a threshold value, IMD 12 may trigger a therapeutic action including, e.g., generating an alarm, modifying one or more parameters by which the vasodilator is programmed to be delivered to patient 16 by IMD 12, and/or switching delivery of the vasodilator from a primary fluid delivery apparatus of IMD 12 to a reserve, e.g., switching delivery from a primary fluid reservoir to a reserve reservoir associated with IMD 12.

Referring again to FIG. 1, in some examples, IMD 12 may also employ pressure sensor 30, which may be configured to sense a pressure in a lumen of a catheter 18 connected to IMD 12. IMD 12 may be configured to analyze the pressure in the lumen of the catheter sensed by pressure sensor 30 to identify one or more catheter malfunctions including, e.g., cuts or occlusions in the catheter. IMD 12 may, in some examples, trigger a therapeutic action when the analysis of the pressure in the lumen of the catheter identifies a catheter malfunction. In one example, IMD 12 generates an alarm and/or switches delivery of the vasodilator from, e.g., a primary fluid reservoir to a reserve reservoir when the analysis of the measured pressure in the lumen of the catheter identifies a catheter malfunction.

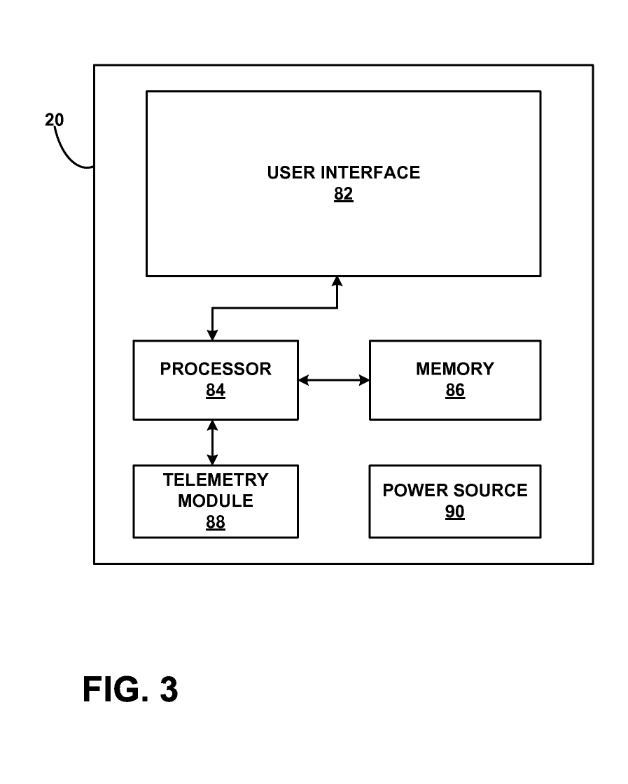

In the example of FIG. 1, IMD 12 delivers a vasodilator to patient 16 from a reservoir within IMD 12 through catheter 18 from a proximal end coupled to IMD 12 to a distal end located proximate to a target delivery site. Catheter 18 can comprise a unitary catheter or a plurality of catheter segments connected together to form an overall catheter length. Additionally, as will be described in detail with reference to FIG. 3, in some examples, IMD 12 may include multiple catheters connected to one or more reservoirs containing the same or different therapeutic fluids. In the example of FIG. 1, IMD 12 includes two catheters 18 and 19. External programmer 20 is configured to wirelessly communicate with IMD 12 as needed, such as to provide or retrieve therapy information or control aspects of therapy delivery (e.g., modify the therapy parameters such as rate or timing of delivery, turn IMD 12 on or off, and so forth) from IMD 12 to patient 16.

IMD 12, in general, may have an outer housing that is constructed of a biocompatible material that resists corrosion and degradation from bodily fluids including, e.g., titanium or biologically inert polymers. IMD 12 may be implanted within a subcutaneous pocket relatively close to the therapy delivery site. For example, in the example shown in FIG. 1, IMD 12 is implanted within the chest of patient 16. In other examples, IMD 12 may be implanted within other suitable sites within patient 16, which may depend, for example, on the target site within patient 16 for the delivery of the therapeutic agent. In still other examples, IMD 12 may be external to patient 16 with a percutaneous catheter connected between IMD 12 and the target delivery site within patient 16.

Catheters 18 and 19 may be coupled to IMD 12 either directly or with the aid of catheter extensions (not shown in FIG. 1). In the example shown in FIG. 1, catheter 18 extends from the implant site of IMD 12 to one or more target delivery sites within patient 16. The target delivery site may depend upon the fluid being delivered by IMD 12. In general, each of catheters 18 and 19 may dispense the same or different drugs in conjunction with or independent of one another at one or more infusion sites within the body of patient 16. In the disclosed examples, both catheters 18 and 19 may be configured to deliver a vasodilator to patient 16 at the same or different delivery sites. In some examples, IMD 12 delivers a vasodilator to a subclavian vein, superior vena cava, or fatty tissue of patient 16 via one or both of catheters 18 and 19. Additional sites to which a vasodilator may be delivered include the renal veins, renal arteries, pulmonary artery and the pericardial sac. Catheters 18 and 19 may be positioned such that one or more fluid delivery outlets (not shown in FIG. 1) of each catheter are proximate to the targets within patient 16.

Although the target sites in the example of FIG. 1 are selected for delivery of a vasodilator to patient 16, therapy system 10 may include alternative target delivery sites for additional applications that are implemented independent of or in conjunction with treating blood pressure via the vasodilator. The target delivery site in other applications of therapy system 10 may be located within patient 16 proximate to, e.g., sacral nerves (e.g., the S2, S3, or S4 sacral nerves) or any other suitable nerve, organ, muscle or muscle group in patient 16, which may be selected based on, for example, a patient condition. In one such application, therapy system 10 may be used to deliver a therapeutic agent, in addition to a vasodilator as shown in FIG. 1, to tissue proximate to a pudendal nerve, a perineal nerve or other areas of the nervous system, in which cases, an additional catheter may be connected to IMD 12 and implanted and substantially fixed proximate to the respective nerve. Positioning a catheter to deliver a therapeutic agent to various sites within patient 16 enables therapy system 10 to assist in managing, e.g., peripheral neuropathy or post-operative pain mitigation, ilioinguinal nerve therapy, intercostal nerve therapy, drug induced gastric stimulation for the treatment of gastric motility disorders and/or obesity, and muscle stimulation, or for mitigation of other peripheral and localized pain (e.g., leg pain or back pain). As another example delivery site, a catheter may be positioned to deliver a therapeutic agent to a deep brain site or within the heart (e.g., intraventricular delivery of the agent). Delivery of a therapeutic agent within the brain may help manage any number of disorders or diseases including, e.g., depression or other mood disorders, dementia, obsessive-compulsive disorder, migraines, obesity, and movement disorders, such as Parkinson's disease, spasticity, and epilepsy. System 10 may also include an additional catheter connected to IMD 12 and positioned to deliver insulin to a patient with diabetes.

Therapy system 10 can be used to, e.g., reduce blood pressure, improve blood flow, cardiac output, renal function and cardiovascular function of patient 16 by delivering a vasodilator to one or more target delivery sites. In such an application, IMD 12 can deliver vasodilator(s) to patient 16 according to one or more dosing programs that set forth different therapy parameters, such as a therapy schedule specifying programmed doses, dose rates for the programmed doses, and specific times to deliver the programmed doses. The dosing programs may be a part of a program group for therapy, where the group includes a plurality of dosing programs and/or therapy schedules. In some examples, IMD 12 may be configured to deliver vasodilator(s) to patient 16 according to different therapy schedules on a selective basis. IMD 12 may include a memory to store one or more therapy programs, instructions defining the extent to which patient 16 may adjust therapy parameters, switch between dosing programs, or undertake other therapy adjustments. Patient 16 or a clinician may select and/or generate additional dosing programs for use by IMD 12 via external programmer 20 at any time during therapy or as designated by the clinician.

In some examples, multiple catheters in addition to catheters 18 and 19 may be coupled to IMD 12 to target the same or different tissue, nerve sites, or blood vessels within patient 16. Thus, although two catheters 18 and 19 are shown in FIG. 1, in other examples, system 10 may include additional catheters for delivering different therapeutic agents to patient 16 and/or for delivering a vasodilator or another therapeutic agent to different tissue sites within patient 16. Accordingly, in some examples, IMD 12 may include a plurality of reservoirs for storing more than one type of therapeutic agent. In some examples, IMD 12 may include a single long tube that contains the therapeutic agent in place of a reservoir. However, an IMD 12 including a primary and reserve reservoir for redundant delivery of a vasodilator to patient 16 is primarily discussed herein with reference to the example of FIG. 1.

Programmer 20 is an external computing device that is configured to communicate with IMD 12 by wireless telemetry. For example, programmer 20 may be a clinician programmer that the clinician uses to communicate with IMD 12. Alternatively, programmer 20 may be a patient programmer that allows patient 16 to view and modify therapy parameters. The clinician programmer may include additional or alternative programming features than the patient programmer. For example, more complex or sensitive tasks may only be allowed by the clinician programmer to prevent patient 16 from making undesired or unsafe changes to the operation of IMD 12.

Programmer 20 may be a hand-held computing device that includes a display viewable by the user and a user input mechanism that can be used to provide input to programmer 20. For example, programmer 20 may include a display screen (e.g., a liquid crystal display or a light emitting diode display) that presents information to the user. In addition, programmer 20 may include a keypad, buttons, a peripheral pointing device, touch screen, voice recognition, or another input mechanism that allows the user to navigate though the user interface of programmer 20 and provide input.

If programmer 20 includes buttons and a keypad, the buttons may be dedicated to performing a certain function, i.e., a power button, or the buttons and the keypad may be soft keys that change in function depending upon the section of the user interface currently viewed by the user. Alternatively, the screen (not shown) of programmer 20 may be a touch screen that allows the user to provide input directly to the user interface shown on the display. The user may use a stylus or their finger to provide input to the display.

In other examples, rather than being a handheld computing device or a dedicated computing device, programmer 20 may be a larger workstation or a separate application within another multi-function device. For example, the multi-function device may be a cellular phone, personal computer, laptop, workstation computer, or personal digital assistant that can be configured with an application to simulate programmer 20. Alternatively, a notebook computer, tablet computer, or other personal computer may enter an application to become programmer 20 with a wireless adapter connected to the personal computer for communicating with IMD 12.

When programmer 20 is configured for use by the clinician, programmer 20 may be used to transmit initial programming information to IMD 12. This initial information may include hardware information for system 10 such as the type of catheter 18 and 19, the position of the catheters within patient 16, the type and amount, e.g., by volume of vasodilator delivered by IMD 12, a refill interval for the therapeutic agent(s), i.e. vasodilator and any additional agents delivered by IMD 12, a baseline orientation of at least a portion of IMD 12 relative to a reference point, therapy parameters of therapy programs stored within IMD 12 or within programmer 20, and any other information the clinician desires to program into IMD 12.

The clinician uses programmer 20 to program IMD 12 with one or more therapy programs that define the therapy delivered by the IMD. During a programming session, the clinician may determine one or more dosing programs that may provide effective therapy to patient 16. In the case of delivering a vasodilator to modulate blood pressure, IMD 12 may provide feedback, e.g. blood pressure or blood flow sensed by sensor 24, to the clinician as to efficacy of a program being evaluated or desired modifications to the program. Once the clinician has identified one or more programs that may be beneficial to patient 16, the evaluation process may continue to determine which dosing program or therapy schedule best alleviates the condition of the patient or otherwise provides efficacious therapy to the patient.

The dosing program information may set forth therapy parameters, such as different predetermined dosages of the therapeutic agent (e.g., a dose amount), the rate of delivery of the therapeutic agent (e.g., rate of delivery of the fluid), the maximum acceptable dose, a time interval between successive supplemental boluses such as patient-initiated boluses (e.g., a lock-out interval), a maximum dose that may be delivered over a given time interval, and so forth. IMD 12 may include a feature that prevents dosing the therapeutic agent in a manner inconsistent with the dosing program. Programmer 20 may assist the clinician in the creation/identification of dosing programs by providing a methodical system of identifying potentially beneficial therapy parameters.

A dosage of a therapeutic agent, such as a drug, may be expressed as an amount of drug, e.g., measured in milligrams or other volumetric units, provided to patient 16 over a time interval, e.g., per day or twenty-four hour period. In this sense, the dosage may indicate a rate of delivery. This dosage amount may convey to the caregiver an indication of the probable efficacy of the drug and the possibility of side effects. In general, a sufficient amount of the drug should be administered in order to have a desired therapeutic effect, such as pain relief. However, the amount of the drug administered to the patient should be limited to a maximum amount, such as a maximum daily dose, in order to avoid potential side effects. Program information specified by a user via programmer 20 may be used to control dosage amount, dosage rate, dosage time, maximum dose for a given time interval (e.g., daily), or other parameters associated with delivery of a drug or other fluid, e.g., a vasodilator by IMD 12.

In some cases, programmer 20 may also be configured for use by patient 16. When configured as the patient programmer, programmer 20 may have limited functionality in order to prevent patient 16 from altering critical functions or applications that may be detrimental to patient 16. In this manner, programmer 20 may only allow patient 16 to adjust certain therapy parameters or set an available range for a particular therapy parameter. In some cases, a patient programmer may permit the patient to control IMD 12 to deliver a supplemental, patient bolus, if permitted by the applicable therapy program administered by the IMD, e.g., if delivery of a patient bolus would not violate a lockout interval or maximum dosage limit. Programmer 20 may also provide an indication to patient 16 when therapy is being delivered or when IMD 12 needs to be refilled or when the power source within programmer 20 or IMD 12 needs to be replaced or recharged.

Whether programmer 20 is configured for clinician or patient use, programmer 20 may communicate to IMD 12 or any other computing device via wireless communication. Programmer 20, for example, may communicate via wireless communication with IMD 12 using radio frequency (RF) telemetry techniques. Programmer 20 may also communicate with another programmer or computing device via a wired or wireless connection using any of a variety of communication techniques including, e.g., RF communication according to the 802.11 or Bluetooth specification sets, infrared (IR) communication according to the IRDA specification set, or other standard or proprietary telemetry protocols. Programmer 20 may also communicate with another programming or computing device via exchange of removable media, such as magnetic or optical disks, or memory cards or sticks including, e.g., non-volatile memory. Further, programmer 20 may communicate with IMD 12 and another programmer via, e.g., a local area network (LAN), wide area network (WAN), public switched telephone network (PSTN), or cellular telephone network, or any other terrestrial or satellite network appropriate for use with programmer 20 and IMD 12.

In accordance with techniques described herein, IMD 12 includes catheters 18 and 19 through which the device delivers a vasodilator to one or more target sites within patient 16 to treat conditions including, e.g., hypertension, heart or kidney failure, and angina. IMD 12 also includes lead 22 to which sensor 24 and electrode 26 are connected. IMD 12 is configured with, e.g., one or more processors or other logical or physical electronic modules to receive at least one of the blood pressure or the blood flow of patient 16 sensed by sensor 24 and trigger a therapeutic action in the event the sensor senses that at least one of blood pressure or blood flow traverses a threshold.

In the example of FIG. 1, sensor 24 and electrode 26 are positioned within right ventricle 28 of heart 14. In other examples, however, system 10 may include one or more sensors arranged in other locations within patient 16 including, e.g., the left ventricle, an atria, the pulmonary artery or a renal vessel of the patient. Sensor 24 is configured to sense at least one of the blood pressure or blood flow of patient 16. In one example, sensor 24 arranged in right ventricle 28 of heart 14 may be configured to sense the pressure in the right ventricle outflow tract (RVOT) from right ventricle 28 through the pulmonary valve to the pulmonary artery. The pressure in right ventricle 28 may be, e.g., a measure of the estimated pulmonary artery diastolic pressure (ePAD) of patient 16. Generally speaking, the pressure needed to open the pulmonary valve of heart 14 is an accurate measure of the pulmonary artery diastolic pressure (PAD), and is commonly referred to as the estimated pulmonary artery diastolic pressure or ePAD.

The ePAD value is a significant pressure value employed in patient monitoring, because ePAD may be used as a basis for evaluating congestive heart failure in a patient. In order to sense ePAD, sensor 24 may, in addition to being arranged in right ventricle 28 as shown in FIG. 1, may also be arranged in the pulmonary artery of heart 14. In other examples, however, sensor 24 may be employed to measure blood pressure values other than ePAD. For example, sensor 24 may be arranged in right ventricle 28 or the pulmonary artery of heart 14 to sense RV systolic or diastolic pressure. Additionally, as noted above, sensor 24 may be configured to sense at least one of blood pressure or blood flow in a renal vessel within patient 16 or in the right atrium to derive estimates of central venous pressures, as a marker of cardiovascular and cardio-renal function. Renal blood pressure may be indicative of one or more renal system conditions including, e.g., kidney failure, impaired glomerular filtration rate, hypertension and end-stage renal dysfunction. A monitoring sensor in the renal vasculature may also serve as a basis for assessing need for and/or effectiveness of dialysis.

In some examples, sensor 24 includes a pressure sensor configured to respond to the absolute pressure inside heart 14 of patient 16. Sensor 24 may be, in such examples, any of a number of different types of pressure sensors. One form of pressure sensor that is useful for measuring blood pressure inside a human heart is a capacitive pressure sensor. Another example pressure sensor is an inductive sensor. In some examples, sensor 24 may also be a piezoelectric or piezoresistive pressure transducer.

In addition to blood pressure, sensor 24 may be configured to sense blood flow of patient 16. In one example, sensor 24 includes a pressure sensor configured to sense blood pressure in one of right ventricle 28, the left ventricle, an atria, the pulmonary artery or a renal vessel of patient 16. IMD 12 may then extrapolate blood flow by integrating the blood pressure of patient 16 over time. In another example, sensor 24 includes an optical blood oxygen saturation sensor configured to measure blood flow of patient 16 as a function of changes in blood oxygen saturation over time. Example optical blood oxygen saturation sensors include pulse oximeters configured to detect changes in light modulation by a body fluid or tissue volume caused by a change in a physiological condition in the body fluid or tissue.

IMD 12 is configured to communicate with sensor 24 via lead 22 to receive sensed blood pressure or blood flow in right ventricle 28 of heart 14, e.g., ePAD of the heart of patient 16. IMD 12 is configured to trigger a therapeutic action in the event sensor 24 senses that at least one of blood pressure or blood flow traverses a threshold. Traversing a threshold, as used in this disclosure, generally refers to exceeding or dropping below the threshold value. As such, blood pressures sensed by sensor 24 that traverse a threshold may either indicate a blood pressure value that is less than or greater than the threshold value. Additionally, the threshold blood pressure or blood flow value may be either a maximum or a minimum blood pressure, which may be stored in, for example, a volatile or non-volatile memory included in IMD 12.

A maximum blood pressure or blood flow threshold may be indicative of the ineffectiveness of the vasodilator to treat patient 16, e.g., because the dosage amount, rate, or frequency are inadequate or inappropriate for the patient. Additionally, the maximum threshold may indicate the ineffectiveness of IMD 12 in delivering the vasodilator to patient 16, e.g., because one or more components of the device are malfunctioning or inoperative. In one example, sensor 24 senses a blood pressure that traverses a maximum blood pressure threshold stored in a memory of IMD 12, i.e., a blood pressure that exceeds a maximum desired blood pressure in this example. The blood pressure of patient 16 sensed by sensor 24 may indicate that the dose of vasodilator delivered to the patient by IMD 12 is ineffective in treating the patient's condition, e.g. hypertension. IMD 12, e.g. a processor of the device may then be configured to generate an alarm indicating that the blood pressure of patient 16 is undesirably high, and, in some examples, the device may also take a remedial measure including, e.g., increasing the dose of vasodilator delivered to the patient.

Conversely, a minimum blood pressure or blood flow threshold value may be indicative of an overdose of vasodilator to patient 16 that acts to reduce the patient's blood pressure or blood flow rate below normal ranges. In one example involving a minimum blood pressure threshold, sensor 24 senses a blood pressure that traverses a minimum blood pressure threshold stored in a memory of IMD 12, i.e., a blood pressure that falls below a desired minimum blood pressure in this example. The blood pressure of patient 16 sensed by sensor 24 may indicate that the dose of vasodilator delivered to the patient by IMD 12 is greater than is necessary to treat the patient's condition, e.g. hypertension, and the current dose is therefore reducing the patient's blood pressure below normal or desirable levels. A processor of IMD 12 may, in such examples, be configured to generate an alarm indicating that the blood pressure of patient 16 is undesirably low, and, in some examples, the device may also take a remedial measure including, e.g., reducing the dose of vasodilator delivered to the patient.

As illustrated in the foregoing examples, in the event sensor 24 senses that blood pressure or blood flow traverses the threshold, IMD 12 is configured to trigger one or more different types of therapeutic actions in response thereto. In one example, IMD 12 is configured as an open loop system in which the device triggers an alarm or other notification in the event the threshold is traversed, but takes no automatic corrective action. For example, IMD 12 may be configured to trigger an audible alert, text-based alert including, e.g., text message or e-mail, or graphical alert regarding the high or low blood pressure or blood flow sensed by sensor 24 by communicating such alert via telemetry to programmer 20 or another electronic device communicatively connected to IMD 12. IMD 12 may also vibrate within patient 16 to alert the patient to the blood pressure or blood flow conditions or cause programmer 20 to vibrate or display a visual alert including, e.g., by emitting light from the programmer. In other examples, in addition to or in lieu of triggering an alarm, IMD 12 may store blood pressure or blood flow sensed by sensor 24 that exceeds or drops below a threshold in, e.g., memory of the device. Stored blood pressure and/or blood flow may be used in conjunction with other techniques to determine if the vasodilator is not effective in treating the condition of patient 16 or that the fluid is not being effectively delivered by IMD 12. For example, IMD 12 may combine the stored blood pressure and/or blood flow sensed by sensor 24 with electrical activity of heart 14 sensed by electrode 26 and/or an activity sensor. Additionally, IMD 12 may combine blood pressure and/or blood flow sensed by sensor 24 with the pressure in the lumen of catheter 18 sensed by pressure sensor 30 and the condition of the catheter as described below.

In other examples, IMD 12 may be configured as a closed loop system in which the device automatically triggers one or more remedial measures in the event sensor 24 indicates that blood pressure or blood flow traverses the threshold. In one example, IMD 12 may be configured to modify one or more parameters by which the device is programmed to deliver the vasodilator to patient 16. For example, IMD 12 may be configured to modify a rate, duration, or frequency of delivery of the vasodilator, or an amount of the vasodilator delivered to the patient when sensor 24 senses that at least one of blood pressure or blood flow traverses a threshold.

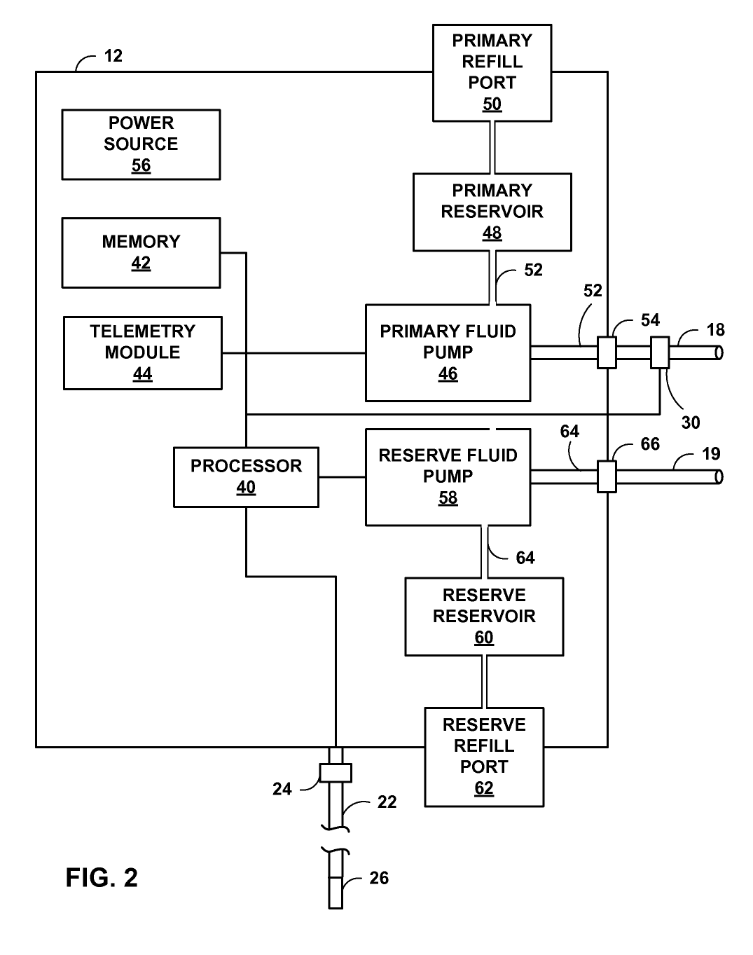

In another example, IMD 12 is configured to switch delivery of the vasodilator from a primary fluid delivery apparatus of IMD 12 to a reserve apparatus when sensor 24 senses that at least one of blood pressure or blood flow traverses a threshold. In some circumstances, an elevated or low blood pressure or blood flow rate in patient 16 may indicate that IMD 12 or some component therein is malfunctioning or inoperative, thereby preventing proper delivery of the vasodilator to the patient. In one example, part or all of the fluid delivery system included in IMD 12, e.g. the fluid pump, valves, fluid conduits, reservoir, and/or refill port may be malfunctioning and causing disruption or complete interruption of the flow of vasodilator to patient 16. In such cases, IMD 12 may be configured to switch from a primary fluid delivery system to a redundant reserve system included with the IMD. The primary and redundant systems may include, e.g., primary and redundant reservoirs that store and dispense an amount of the vasodilator. As illustrated in FIG. 2, however, in another example, the redundant reserve system may include an entire fluid delivery apparatus of IMD 12 including a reserve pump, reservoir, and refill port.

In other examples, a processor of IMD 12 may be configured to generate an alert and trigger one or more remedial measures in the event that sensor 24 senses blood pressure or blood flow that traverses a threshold.

As illustrated in the example of FIG. 1, catheter 18 may also include pressure sensor 30 configured to sense a pressure in a lumen of the catheter. In some examples disclosed herein, IMD 12 is configured to analyze the pressure in the lumen of catheter 18 sensed by pressure sensor 30 to identify one or more catheter malfunctions including, e.g., cuts or occlusions in the catheter. Pressure sensor 30 and the analysis of the pressure in the lumen of catheter 18 may be controlled independent of or in conjunction with the blood pressure or blood flow measurements made by sensor 24. In one example, pressure sensor 30 may be controlled to sense a pressure in the lumen of catheter 18 in the event sensor 24 senses that blood pressure or blood flow traverses the threshold. In another example, pressure sensor 30 may continuously or periodically sense the pressure in the lumen of catheter 18 independent of any blood pressure of blood flow sensing by sensor 24.

During operation, IMD 12 may deliver fluid in controlled pulses. When IMD 12 delivers a fluid dose through catheter 18 to patient 16, the device may also control pressure sensor 30 to measure a pressure pulse within a lumen of catheter 18 that is generated by the delivery of fluid through the lumen. Pressure sensor 30 can also measure a steady state baseline pressure within the lumen of catheter 18 when no fluid dose is being delivered to patient 16. Pressure sensor 30 can be any of a number of types of sensors that are capable of measuring the pressure within a lumen of an implantable catheter including, e.g. capacitive, piezoelectric, piezoresistive, or inductive pressure sensors.

In some circumstances, catheter 18 may become disconnected from IMD 12 or otherwise malfunction due to, e.g., cuts or occlusions in the catheter. IMD 12 can therefore discern whether one or more characteristics of the pressure pulse within the lumen of catheter 18 measured by pressure sensor 30 is indicative of a catheter malfunction. For example, IMD 12 can determine if the maximum pressure of the pressure pulse measured by pressure sensor 30 is below a minimum pressure threshold value, which may indicate the presence of an air bubble in the fluid pathway or that catheter 18 is disconnected completely from IMD 12. Additionally, IMD 12 can determine if the decay time of the pressure pulse is below a minimum threshold value, which may indicate a leak in catheter 18. In another example, IMD 12 can determine if the decay time is above a maximum threshold value, which may indicate an occlusion in catheter 18. IMD 12 can also analyze the pressure pulse measured by pressure sensor 30 by determining if the pressure within the lumen of catheter 18 falls below a baseline pressure after decaying from a maximum pressure, which may indicate either a cut in the catheter or that the catheter is disconnected from IMD 12. An expanded explanation of identifying catheter malfunctions from measured pressure pulses may be found in commonly assigned U.S. Patent Publication No. 2007/0270782 A1, entitled SYSTEMS AND METHODS OF IDENTIFYING CATHETER MALFUNCTIONS USING PRESSURE SENSING, by Miesel et al., published Nov. 22, 2007, the entire content of which is incorporated herein by this reference.

In some examples, IMD 12 may be configured to employ pressure sensor 30 as an additional test of the operation of the device when sensor 24 senses that at least one of blood pressure or blood flow traverses a threshold. In other examples, IMD 12 may be configured to analyze the pressure within the lumen of catheter 18 sensed by pressure sensor 30 independent of any blood pressure or blood flow information gleaned from sensor 24. In any event, IMD 12 may, in some examples, trigger a therapeutic action when the analysis of the pressure in the lumen of catheter 18 identifies a catheter malfunction. In one example, IMD 12 generates an alarm and/or switches delivery of the vasodilator from, e.g., a primary fluid reservoir connected to catheter 18 to a reserve reservoir connected to reserve catheter 19, when the analysis of the pressure in the lumen of catheter 18 identifies a catheter malfunction.

IMD 12, in the example of FIG. 1, is also coupled to electrode 26 located at the distal end of lead 22 near sensor 24 in right ventricle 28. In some examples, electrode 26 takes the form of an extendable helix tip electrode mounted retractably within an insulative electrode head at the distal end of lead 22. In other examples, electrode 26 is a small circular electrode at the tip of a tined lead or other fixation element. Electrode 26 may be employed as a pair of electrodes connected to lead 22 and configured for bipolar sensing or in combination with an electrode connected to or a part of the housing of IMD 12 for unipolar sensing of the electrical activity of heart 14. Electrode 26 may be controlled by IMD 12, e.g. via a sensing module, to sense electrical activity in heart 12. In one example, IMD 12 senses R-waves of heart 12 via electrode 26. IMD 12 may calculate a rate of heart 14 as a function of R-R intervals collected via R-wave sensing, e.g., on a beat-to-beat continuous basis. Based on the calculated heart rate, it may be determined if the patient is at rest or, e.g. performing any of a number of activities that may cause elevated heart rates. Heart rate calculation may be augmented by activity sensing via an activity sensor included in separate from IMD 12. In one example, IMD 12 includes an activity sensor in the form of a single or multi-axis accelerometer that generates signals that vary as a function of a measured parameter relating to the patient's metabolic requirements, activity level, and/or posture. Based on the output of the activity sensor and/or the calculated heart rate, IMD 12 may determine if patient 16 is at rest, as indicated by minimal activity sensor output, or performing activities, as indicated by significant activity sensor output and elevated heart rates.

The rate of heart 14 and activity of patient 16 may be employed by IMD 12 in the examples disclosed herein to augment or otherwise inform blood pressure and/or blood flow measurements made via sensor 24 (and/or other sensors arranged within patient 16) or any therapeutic action triggered by IMD 12 from the blood pressure measurements. For example, IMD 12 may be programmed with one blood pressure or blood flow threshold value for periods of time in which patient 16 is not active, and a higher threshold value for periods of activity. Additionally, IMD 12 may generally reference patient activity via the activity sensor or rate of heart 14 as an additional check of patient condition in the event sensor 24 indicates a blood pressure or blood flow value that is, e.g., greater than a threshold value.

FIG. 2 is a functional block diagram illustrating components of an example of IMD 12, which includes pressure sensor 30, processor 40, memory 42, telemetry module 44, primary fluid pump 46, primary reservoir 48, primary refill port 50, internal tubing 52, catheter access port 54, and power source 56. IMD 12 also includes a redundant fluid delivery apparatus including reserve fluid pump 58, reservoir 60, and refill port 62, and internal tubing 64 and catheter access port 66. Processor 40 is communicatively connected to memory 42, telemetry module 44 and primary and reserve fluid pumps 46, 58. Primary fluid delivery pump 46 is connected to primary reservoir 48 via internal tubing 52. Primary reservoir 48 is connected to primary refill port 50. Catheter access port 54 is connected to internal tubing 52 and catheter 18. Reserve fluid delivery pump 58 is connected to reserve reservoir 60 via internal tubing 64. Reserve reservoir 60 is connected to reserve refill port 62. Catheter access port 66 is connected to internal tubing 64 and catheter 19. IMD 12 also includes power source 56, which is configured to deliver operating power to various components of the IMD.

During normal operation of IMD 12, processor 40 controls primary fluid pump 46 with the aid of instructions associated with program information that is stored in memory 42 to deliver a vasodilator to patient 16 via catheter 18. Instructions executed by processor 40 may, for example, define dosing programs that specify the amount of vasodilator that is delivered to a target tissue site within patient 16 from primary reservoir 48 via catheter 18. The instructions may further specify the time at which the agent will be delivered and the time interval over which the agent will be delivered. The amount of the agent and the time over which the agent will be delivered are a function of, or alternatively determine, the dosage rate at which the fluid is delivered. The therapy programs may also include other therapy parameters, such as the frequency of bolus delivery, the type of therapeutic agent delivered if IMD 12 is configured to deliver more than one type of therapeutic agent, and so forth. Components described as processors within IMD 12, external programmer 20, or any other device described in this disclosure may each comprise one or more processors, such as one or more microprocessors, digital signal processors (DSPs), application specific integrated circuits (ASICs), field programmable gate arrays (FPGAs), programmable logic circuitry, or the like, either alone or in any suitable combination. Additionally, the functions attributed to processors described herein may be implemented in one or more logical or physical modules. For example, the pressure sensing and analyses functions described with reference to IMD 12 may, in some examples, be implemented in a logical or physical pressure monitor module in the device, while other functions including, e.g., therapy delivery may be implemented in one or more separate modules.

Upon instruction from processor 40, primary fluid pump 46 draws fluid from primary reservoir 48 and pumps the fluid through internal tubing 52 to catheter 18 through which the vasodilator is delivered to patient 16 to effect one or more of the treatments described above. Internal tubing 52 is a segment of tubing or a series of cavities within IMD 12 that run from primary reservoir 48, around or through primary fluid pump 46 to catheter access port 54. Primary fluid pump 46 can be any mechanism that delivers a therapeutic agent in some metered or other desired flow dosage to the therapy site within patient 16 from reservoir 48 via implanted catheter 18.

In one example, primary fluid pump 46 can be a squeeze pump that squeezes internal tubing 52 in a controlled manner, e.g., such as a peristaltic pump, to progressively move fluid from primary reservoir 48 to the distal end of catheter 18 and then into patient 16 according to parameters specified by a set of program information stored on memory 42 and executed by processor 40. Primary fluid pump 46 can also be an axial pump, a centrifugal pump, a pusher plate, a piston-driven pump, or other means for moving fluid through internal tubing 52 and catheter 18. In one particular example, primary fluid delivery pump 46 can be an electromechanical pump that delivers fluid by the application of pressure generated by a piston that moves in the presence of a varying magnetic field and that is configured to draw fluid from primary reservoir 48 and pump the fluid through internal tubing 52 and catheter 18 to patient 16.

Periodically, fluid may need to be supplied percutaneously to primary reservoir 48 because all of a therapeutic agent has been or will be delivered to patient 16, or because a clinician wishes to replace an existing agent with a different agent or similar agent with different concentrations of therapeutic ingredients. Primary refill port 50 can therefore comprise a self-sealing membrane to prevent loss of therapeutic agent delivered to primary reservoir 48 via the primary refill port. For example, after a percutaneous delivery system, e.g., a hypodermic needle, penetrates the membrane of primary refill port 50, the membrane may seal shut when the needle is removed from the refill port.

In the event the redundant fluid delivery apparatus of IMD 12 is activated by processor 40 in examples described in this disclosure, reserve fluid pump 58, reservoir 60, refill port 62, and internal tubing 64 and catheter access port 66 function to deliver a vasodilator fluid agent to patient 16 via catheter 19 in the same manner as described above with reference to primary fluid pump 46, reservoir 48, refill port 50, and internal tubing 52 and catheter access port 54 delivering the therapeutic fluid to the patient via catheter 18.

In some examples, processor 40 of IMD 12 is configured to receive blood pressure or blood flow in right ventricle 28 of heart 14 sensed by sensor 24 via lead 22 indicative of, e.g., ePAD of the heart of patient 16. Processor 40 may be configured to trigger a therapeutic action in the event that sensor 24 senses that at least one of blood pressure or blood flow traverses a threshold. As noted above, the threshold blood pressure or blood flow value may represent either a maximum or a minimum threshold value. The threshold blood pressure or blood flow value or values employed by processor 40 as bases to trigger therapeutic action may be, for example, stored in memory 42 of IMD 12.

In the event sensor 24 senses that blood pressure or blood flow traverses the threshold, processor 40 may be configured to trigger one or more different types of therapeutic actions in response thereto. In one example, IMD 12 is configured as an open loop system in which processor 40 is programmed to trigger an alarm in the event the threshold is traversed, but takes no automatic corrective action. For example, processor 40 may be configured to trigger an audible, visual, or tactile alert regarding blood pressure or blood flow sensed by sensor 24 that traverses a threshold in the manner described above.

In other examples, IMD 12 is configured as a closed loop system in which processor 40 automatically triggers one or more remedial measures in the event sensor 24 senses blood pressure or blood flow that traverses the threshold. In one example, processor 40 is configured to modify one or more parameters by which the device is programmed to deliver the vasodilator to patient 16. In one example, processor 40 is configured to control IMD 12 to deliver the vasodilator to patient 16 according to a dosing program and/or therapy schedule stored in memory 42, which includes, e.g., delivery rate, duration, frequency, etc. Processor 40 may be configured, in such examples, to modify a rate, duration, or frequency of delivery of the vasodilator, or an amount of the vasodilator delivered to patient 16 by IMD 12 when sensor 24 senses at least one of blood pressure or blood flow that traverses a threshold. Processor 40 may, in some examples, resort to the modified therapies for a temporary period of time determined based on feedback from sensor 24, i.e., blood pressure or blood flow within acceptable ranges. In other examples, processor 40 may temporarily or permanently modify the dosing program and/or therapy schedule stored in memory 42 and according to which IMD 12 delivers the vasodilator to patient 16.

In another example, IMD 12 is configured to switch delivery of the vasodilator from a primary fluid delivery apparatus of IMD 12 to a reserve apparatus when sensor 24 senses that at least one of blood pressure or blood flow traverses a threshold. In some circumstances, an elevated or low blood pressure or blood flow rates in patient 16 may indicate that some component of IMD 12 is malfunctioning or inoperative, thereby preventing proper delivery of the vasodilator to the patient. In one example, part or all of the fluid delivery system included in IMD 12, e.g. primary fluid pump 46, internal tubing 52, reservoir 48, and/or refill port 50 may be malfunctioning and causing disruption or complete interruption of the flow of vasodilator to patient 16. In such cases, processor 40 may be configured to switch from the primary fluid delivery system including primary fluid pump 46, reservoir 48, refill port 50, internal tubing 52, and catheter access port 54 connected to catheter 18 to the redundant reserve system including reserve fluid pump 58, reservoir 60, refill port 62, internal tubing 64, and catheter access port 66 connected to catheter 19. Although the example of FIG. 2, shows a redundant reserve system including an entire fluid delivery apparatus, other examples may include, e.g., a primary and redundant system comprising only primary and redundant reservoirs that store and dispense an amount of the vasodilator to patient 16 via a common fluid pump. In one such example, a primary and reserve reservoir each include respective primary and reserve refill ports as with the example of FIG. 2. However, the primary and reserve reservoirs are both connected to a single pump by, e.g., a valve that may be controlled by processor 40 to switch delivery of the vasodilator to the patient by the fluid pump from the primary to the reserve reservoir.

Catheter 18 may also include pressure sensor 30 configured to sense a pressure in a lumen of the catheter. In some examples disclosed herein, processor 40 is configured to analyze the pressure in the lumen of catheter 18 sensed by pressure sensor 30 to identify one or more catheter malfunctions including, e.g., cuts or occlusions in the catheter in the manner described above with reference to FIG. 1. Additionally, as noted above with reference to FIG. 1, IMD 12 may be configured to employ pressure sensor 30 as an additional test of the operation of the device when sensor 24 senses that at least one of blood pressure or blood flow traverses a threshold. In other examples, IMD 12 may be configured to analyze the pressure within the lumen of catheter 18 sensed by pressure sensor 30 independent of any blood pressure or blood flow information gleaned from sensor 24. In any event, IMD 12 may, in some examples, trigger a therapeutic action when the analysis of the pressure in the lumen of catheter 18 by processor 40 identifies a catheter malfunction

Processor 40, or a sensing module controlled by processor 40 may also control electrode 26 connected to lead 22 to sense electrical activity in heart 12. In one example, processor 40 controls electrode 26 to sense R-waves of heart 12. Processor 40 may calculate a rate of heart 14 as a function of R-R intervals collected via R-wave sensing. Based on the calculated heart rate, it may be determined if the patient is at rest or, e.g. performing any of a number of activities that may cause elevated heart rates. Heart rate calculation may be augmented by activity sensing via an activity sensor included in or separate from IMD 12. In one example, IMD 12 includes an activity sensor in the form of a single or multi-axis accelerometer that generates signals that vary as a function of a measured parameter relating to the patient's metabolic requirements, activity level, and/or posture. In some examples, the activity sensor may be connected to processor 40 and may store activity signals in memory 42. Based on the output of the activity sensor and/or the calculated heart rate, processor 40 may determine if patient 16 is at rest, as indicated by minimal activity sensor output, or performing activities, as indicated by significant activity sensor output and elevated heart rates. The rate of heart 14 and activity of patient 16 may be employed by processor 40 in the examples disclosed herein to augment or otherwise inform blood pressure or blood flow measurements made via sensor 24 (and/or other sensors arranged within patient 16) or any therapeutic action triggered by processor 40 therefrom.

Although triggering therapeutic actions when one or more of sensor 24 senses that blood pressure or blood flow traverses a threshold or pressure sensor 30 identifies a catheter malfunction has been described as executed by IMD 12 and, in particular, processor 40, in other examples one or more of these functions may be carried out by other devices including, e.g., external programmer 20. For example, one or both of blood pressure or blood flow sensed by sensor 24 and/or catheter lumen pressure sensed by pressure sensor 30 may be communicated from IMD 12 via telemetry module 44 to programmer 20. The parameters may be analyzed by a processor of programmer 20 to determine if, e.g., sensor 24 senses at least one of blood pressure or blood flow that traverses a threshold stored in a memory of the programmer. In this example, programmer 20 may then communicate with IMD 12 via telemetry module 44 and processor 40 or a processor of the programmer may trigger a therapeutic action as described above in response to the high or low blood pressure or blood flow sensed by sensor 24. For example, programmer 20 may generate an audible, visual, or tactile alert.

In addition to storing information sensed by sensors 24 and 30 and blood pressure and/or blood flow thresholds, memory 28 of IMD 12 may store program information including instructions for execution by processor 26, such as, but not limited to, therapy programs, historical therapy programs, timing programs for delivery of fluid from primary reservoir 34 to catheter 18, and any other information regarding therapy of patient 16. A program may indicate the bolus size or flow rate of the drug, and processor 26 may accordingly deliver therapy. A program may also indicate the frequency at which sensor 24 is configured to sense blood pressure or blood flow of patient 16 and pressure sensor 30 is commanded to measure a pressure pulse within catheter 18. Memory 28 may include separate memories for storing instructions, patient information, therapy parameters (e.g., grouped into sets referred to as “dosing programs”), therapy adjustment information, program histories, and other categories of information such as any other data that may benefit from separate physical memory modules. Therapy adjustment information may include information relating to timing, frequency, rates and amounts of patient boluses or other permitted patient or automatic device controlled modifications to therapy. In some examples, memory 28 stores program instructions that, when executed by processor 26, cause IMD 12 and processor 26 to perform the functions attributed to them in this disclosure.

At various times during the operation of IMD 12 to treat patient 16, communication to and from IMD 12 may be necessary to, e.g., change therapy programs, adjust parameters within one or more programs, configure or adjust a particular bolus, send or receive high blood pressure or blood flow alert, or to otherwise download information to or from IMD 12. Processor 26 therefore controls telemetry module 30 to wirelessly communicate between IMD 12 and other devices including, e.g. programmer 20. Telemetry module 30 in IMD 12, as well as telemetry modules in other devices described herein, such as programmer 20, can be configured to use RF communication techniques to wirelessly send and receive information to and from other devices respectively. In addition, telemetry module 30 may communicate with programmer 20 via proximal inductive interaction between IMD 12 and the external programmer. Telemetry module 30 may send information to external programmer 20 on a continuous basis, at periodic intervals, or upon request from the programmer.

Power source 44 delivers operating power to various components of IMD 12. Power source 44 may include a small rechargeable or non-rechargeable battery and a power generation circuit to produce the operating power. In the case of a rechargeable battery, recharging may be accomplished through proximal inductive interaction between an external charger and an inductive charging coil within IMD 12. In some examples, power requirements may be small enough to allow IMD 12 to utilize patient motion and implement a kinetic energy-scavenging device to trickle charge a rechargeable battery. In other examples, traditional batteries may be used for a limited period of time. As another alternative, an external inductive power supply could transcutaneously power IMD 12 as needed or desired.

FIG. 3 is a functional block diagram illustrating various components of external programmer 20 for IMD 12. As shown in FIG. 3, external programmer 20 includes user interface 82, processor 84, memory 86, telemetry module 88, and power source 90. A clinician or patient 16 interacts with user interface 82 in order to manually change the parameters of a dosing program, change dosing programs within a group of programs, view therapy information, view historical therapy regimens, establish new therapy regimens, or otherwise communicate with IMD 12 or view or edit programming information.

User interface 82 may include a screen and one or more input buttons, as discussed in greater detail below, that allow external programmer 20 to receive input from a user. Alternatively, user interface 82 may additionally or only utilize a touch screen display, as in the example of clinician programmer 60. The screen may be a liquid crystal display (LCD), dot matrix display, organic light-emitting diode (OLED) display, touch screen, or any other device capable of delivering and/or accepting information. For visible indications of therapy program parameters or operational status, a display screen may suffice. For audible and/or tactile indications of therapy program parameters or operational status, programmer 20 may further include one or more audio speakers, voice synthesizer chips, piezoelectric buzzers, or the like.

Input buttons for user interface 82 may include a touch pad, increase and decrease buttons, emergency shut off button, and other buttons needed to control the therapy, as described above with regard to patient programmer 20. Processor 84 controls user interface 82, retrieves data from memory 86 and stores data within memory 86. Processor 84 also controls the transmission of data through telemetry module 88 to IMD 12. The transmitted data may include therapy program information specifying various drug delivery program parameters. Memory 86 may include operational instructions for processor 84 and data related to therapy for patient 16.

User interface 82 may be configured to present therapy program information to the user. User interface 82 enables a user to program IMD 12 in accordance with one or more dosing programs, therapy schedules, or the like. For example, a user such as a clinician, physician or other caregiver may input patient information, drug information including therapy schedules, priming information, bridging information, drug/IMD implant location information, or other information to programmer 20 via user interface 82. In addition, user interface 82 may display therapy program information as graphical bar graphs or charts, numerical spread sheets, or in any other manner in which information may be displayed. Further, user interface 82 may present nominal or suggested therapy parameters that the user may accept via user interface 82.

As described above, one or more of the functions attributed to IMD 12, and, in particular, processor 40, may be performed instead by or in conjunction with programmer 20. For example, processor 84 of programmer 20 may be employed to receive blood pressure or blood flow sensed by sensor 24 and/or catheter lumen pressure sensed by pressure sensor 30, communicate with IMD 12 to trigger therapeutic actions, and/or receive commands from IMD 12 to execute a therapeutic action like generating an alert for a user regarding a high or low blood pressure or blood flow of patient 16. Programmer 20, and, in particular, processor 84 may execute these functions instead of or in conjunction with one or more components of IMD 12 including, e.g., processor 40, memory 42, and telemetry module 44. In other words, processor 84 may, for example, communicate with IMD 12 via telemetry module 88 of programmer 20 and telemetry module 44 of IMD 12 to directly control the fluid delivery system components (pump 46 or 58) of IMD 12 to, e.g., switch from delivering the vasodilator from primary reservoir 48 via pump 46 to delivering the drug from reserve reservoir 60 via pump 58. Alternatively, processor 84 may communicate with processor 40, which in turn controls the fluid delivery system components of IMD 12 in response to commands from processor 84.

Telemetry module 88 allows the transfer of data to and from IMD 12. Telemetry module 88 may communicate automatically with IMD 12 at a scheduled time or when the telemetry module detects the proximity of IMD 12. Alternatively, telemetry module 88 may communicate with IMD 12 when signaled by a user through user interface 82 of programmer 20. To support RF communication, telemetry module 88 may include appropriate electronic components, such as amplifiers, filters, mixers, encoders, decoders, and the like. Power source 90 may be a rechargeable battery, such as a lithium ion or nickel metal hydride battery. Other rechargeable or conventional batteries may also be used. In some cases, external programmer 20 may be used when coupled to an alternating current (AC) outlet, i.e., AC line power, either directly or via an AC/DC adapter.

In some examples, external programmer 20 may be configured to recharge IMD 12 in addition to programming IMD 12. Alternatively, a recharging device may be capable of communication with IMD 12. Then, the recharging device may be able to transfer programming information, data, or any other information described herein to IMD 12. In this manner, the recharging device may be able to act as an intermediary communication device between external programmer 20 and IMD 12. Generally speaking, the techniques for triggering therapeutic actions based on parameters sensed by sensors 24 and 30 described in this disclosure may be distributed between IMD 12 and any type of external device capable of communication therewith.

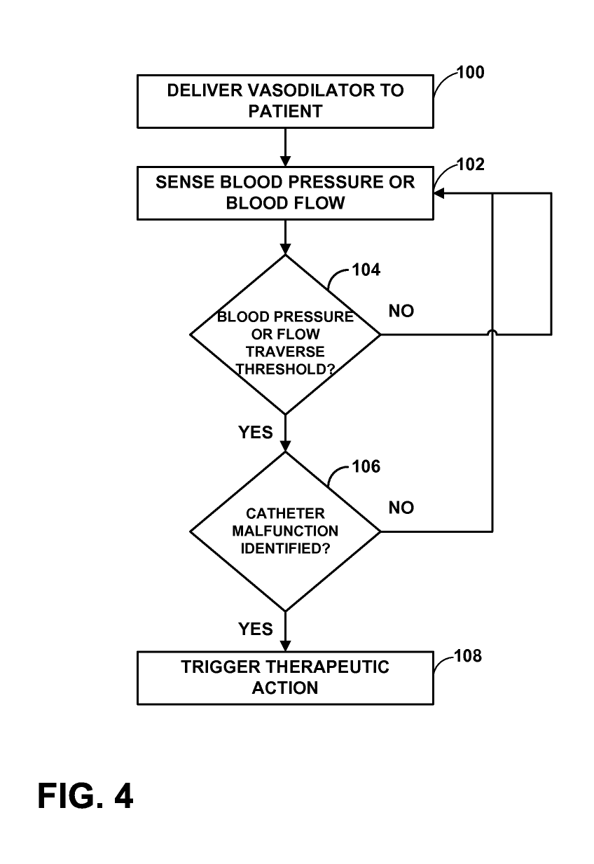

FIG. 4 is a flow chart illustrating an example method of triggering therapeutic action in response to a patient blood pressure or blood flow that traverses a threshold value in a patient receiving a vasodilator delivered by a fluid delivery device. The method illustrated in FIG. 4 includes delivering a vasodilator to a patient with a fluid delivery device (100), sensing at least one of blood pressure or blood flow (102), determining if blood pressure or blood flow traverses a threshold (104), optionally determining if one or more catheter malfunctions are identified (106), and triggering a therapeutic action when ate least one of sensed blood pressure or blood flow traverses a threshold and, optionally, one or more catheter malfunctions are identified (108). The method of FIG. 4 is described below in the context of IMD 12, and, in particular, processor 40 of IMD 12 performing the functions included therein. However, as described above, one or more of the functions included in the method of FIG. 4 and attributed to IMD 12 may be performed by another electronic device including, e.g., programmer 20 and/or other devices communicatively connected to programmer 20 and/or IMD 12.

The method of FIG. 4 includes delivering a vasodilator to a patient with a fluid delivery device (100). In one example, IMD 12 is configured to deliver a vasodilator to one or more target sites within patient 16 via catheter 18 and/or catheter 19 to treat conditions including, e.g., hypertension, heart and kidney failure, and angina. IMD 12 delivers a vasodilator to patient 16 from a reservoir within IMD 12 through catheter 18 to a target delivery site.

The method of FIG. 4 also includes sensing at least one of blood pressure or blood flow (102). In one example, IMD 12 is coupled to sensor 24 via lead 22 positioned within right ventricle 28 of heart 14, as shown in FIG. 1. In other examples, however, one or more sensors coupled to IMD 12 may be arranged in other locations within patient 16 including, e.g., the left ventricle, an atria, the pulmonary artery or a renal vessel of the patient. Sensor 24 is configured to sense at least one of the blood pressure or blood flow of patient 16. For example, sensor 24 arranged in right ventricle 28 of heart 14 may be configured to sense the pressure in the right ventricle outflow tract from right ventricle 28 through the pulmonary valve to the pulmonary artery. The pressure in right ventricle 28 may be, e.g., a measure of the estimated pulmonary artery diastolic pressure (ePAD) of patient 16. In other examples, however, sensor 24 may be employed to measure blood pressure in other locations or blood flow rates. For example, sensor 24 may be configured to sense blood pressure or blood flow in a renal vessel within patient 16.

Processor 40 of IMD 12 is connected to and configured to receive blood pressure and/or blood flow sensed by sensor 24 via lead 22. Processor 40 may temporarily employ the blood pressure or blood flow sensed by sensor 24 and/or may store blood pressures or blood flow rates in memory 42 of IMD 12.

In some examples, IMD 12 employs pressure sensor 30 that is configured to sense a pressure in a lumen of a catheter connected to IMD 12. Processor 40 may, in some examples, be configured to analyze the pressure in the lumen of catheter 18 sensed by pressure sensor 30 to identify one or more catheter malfunctions including, e.g., cuts or occlusions in the catheter. Processor 40 may temporarily employ the catheter lumen pressures sensed by pressure sensor 30 and/or may store the pressures and any associated malfunctions identified therefrom in memory 42 of IMD 12.

In addition sensing at least one of blood pressure or blood flow with sensor 24 and sensing catheter lumen pressure with pressure sensor 30 (102), the method of FIG. 4 also includes determining if blood pressure or blood flow sensed by sensor 24 traverses a threshold (104). In some examples, processor 40 of IMD 12 is configured to receive blood pressure or blood flow in right ventricle 28 of heart 14 sensed by sensor 24 (102) via lead 22. Processor 40 may then determine if at least one of blood pressure or blood flow sensed by sensor 24 traverses a threshold (104). The threshold blood pressure or blood flow value may be either a maximum or a minimum. The threshold blood pressure or blood flow value or values with which processor 40 compares the blood pressure or blood flow sensed by sensor 24 may be, for example, stored in memory 42 of IMD 12.

The method of FIG. 4 optionally includes determining if one or more catheter malfunctions are identified (106) based on the pressure in a lumen of catheter 18 sensed by pressure sensor 30. During operation of IMD 12 to deliver a vasodilator to patient 16, processor 40 may control primary fluid pump 46 to deliver the vasodilator in controlled pulses from primary reservoir 48 to patient 16 via internal tubing 52, catheter access port 54, and catheter 18. When IMD 12 delivers a fluid dose through catheter 18 to patient 16, processor 40 may also control pressure sensor 30 to measure a pressure pulse within a lumen of catheter 18 that is generated by the delivery of fluid therethrough. Processor 40 can analyze the pressure in the lumen of catheter 18 sensed by pressure sensor 30 to determine whether one or more characteristics of the pressure pulse within the lumen is indicative of a catheter malfunction.

In the event one or both of blood pressure or blood flow sensed by sensor 24 traverses the threshold or pressure sensor 30 identifies a catheter malfunction, IMD 12 is configured to trigger one or more therapeutic actions in response thereto (108). In one example, IMD 12 is configured as an open loop system in which processor 40 is programmed to trigger an alarm in the event the threshold is traversed and, optionally, a catheter malfunction is identified. For example, processor 40 may be configured to trigger an audible, visual, or tactile alert regarding blood pressure or blood flow sensed by sensor 24 that traverses the threshold.

In other examples, IMD 12 may be configured as a closed loop system in which processor 40 automatically triggers one or more remedial measures in the event sensor 24 senses that at least one of blood pressure or blood flow traverses a threshold and, optionally, pressure sensor 30 identifies a catheter malfunction. In one example, processor 40 is configured to modify one or more parameters by which the device is programmed to deliver the vasodilator to patient 16. Processor 40 may, in some examples, resort to the modified therapies for a temporary period of time determined based on feedback from sensor 24, i.e. blood pressure or blood flow within acceptable ranges. In other examples, processor 40 may temporarily or permanently modify the dosing program and/or therapy schedule stored in memory 42 and according to which IMD 12 delivers the vasodilator to patient 16. In another example, IMD 12 is configured to switch delivery of the vasodilator from a primary fluid delivery apparatus of IMD 12 to a reserve apparatus when sensor 24 senses that at least one of blood pressure or blood flow traverses a threshold, and, optionally, pressure sensor 30 identifies a catheter malfunction.

In some examples, IMD 12 is configured to generate an alert and trigger one or more remedial measures in the event sensor 24 senses that blood pressure or blood flow traverses the threshold (e.g., exceeds a maximum threshold or drops below a minimum threshold, in alternate examples) and, optionally, pressure sensor 30 identifies a catheter malfunction.

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied a computer-readable medium, such as a computer-readable storage medium, containing instructions for execution by a processor. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described in this disclosure. These and other examples are within the scope of the following claims.

1. A fluid delivery system comprising:
a fluid delivery device configured to deliver a vasodilator;
a sensor configured to sense at least one of blood pressure or blood flow in one of a ventricle or an atria of a heart, a pulmonary artery, and a renal vessel; and
a processor configured to trigger a therapeutic action when the sensed at least one of blood pressure or blood flow traverses a threshold.

2. The system of claim 1, wherein the therapeutic action comprises triggering an alarm when the sensor senses that the at least one of blood pressure or blood flow traverses the threshold.

3. The system of claim 2, wherein the alarm comprises one of an audible, tactile, or visual alert.

4. The system of claim 2, wherein the processor is configured to cause the fluid delivery device to vibrate when the sensed at least one of blood pressure or blood flow traverses the threshold.

5. The system of claim 1, wherein the therapeutic action comprises modifying at least one of a rate, duration, or frequency of delivery of the vasodilator or an amount of the vasodilator delivered when the sensed at least one of blood pressure or blood flow traverses the threshold.

6. The system of claim 1 further comprising:
a primary reservoir configured to receive and store a first amount of the vasodilator; and
a reserve reservoir configured to receive and store a second amount of the vasodilator,
wherein the fluid delivery device is configured to deliver the vasodilator via one or both of the primary reservoir and the reserve reservoir.

7. The system of claim 6, wherein the therapeutic action comprises activating the reserve reservoir to deliver the vasodilator when the sensed at least one of blood pressure or blood flow traverses the threshold.

8. The system of claim 1 further comprising:
a catheter connected to the fluid delivery device;
a pressure sensor configured to sense a pressure in a lumen of the catheter; and
the processor configured to analyze the pressure in the lumen of the catheter to identify one or more catheter malfunctions.

9. The system of claim 8, wherein the processor is configured to trigger the therapeutic action when the analysis of the pressure in the lumen of the catheter identifies one or more catheter malfunctions.

10. The system of claim 1, wherein the fluid delivery device comprises the processor.

11. The system of claim 1 further comprising a programmer that comprises the processor, wherein the programmer is configured to program the fluid delivery device.

12. The system of claim 1, wherein the sensor comprises at least one of a pressure or an optical blood oxygen saturation sensor.

13. A fluid delivery system comprising:
a primary fluid delivery apparatus configured to deliver a vasodilator;
a reserve fluid delivery apparatus configured to deliver the vasodilator;
a sensor configured to sense at least one of blood pressure or blood flow in one of a ventricle or an atria of a heart, a pulmonary artery, and a renal vessel; and
a processor configured to switch delivery of the vasodilator from the primary delivery apparatus to the reserve fluid delivery apparatus when the sensed at least one of blood pressure or blood flow traverses the threshold.

14. The fluid delivery system of claim 13, wherein at least one of the primary fluid delivery apparatus or the reserve fluid delivery apparatus comprises at least one of a fluid pump or a fluid reservoir.

15. The fluid delivery system of claim 13, wherein the processor is configured to switch delivery of the vasodilator from a primary fluid reservoir included in the primary delivery apparatus to a reserve fluid reservoir included in the reserve fluid delivery apparatus when the sensed at least one of blood pressure or blood flow traverses the threshold.

16. The system of claim 13, wherein the processor is configured to trigger an alarm when the sensed at least one of blood pressure or blood flow traverses the threshold.

17. The system of claim 16, wherein the alarm comprises one of an audible, tactile, or visual alert.

18. The system of claim 16, wherein the processor is configured to cause at least a portion of the fluid delivery system to vibrate when the sensed at least one of blood pressure or blood flow traverses the threshold.

19. The system of claim 13, wherein the processor is configured to modify at least one of a rate, duration, or frequency of delivery of the vasodilator or an amount of the vasodilator delivered when the sensed at least one of blood pressure or blood flow traverses the threshold.

20. The system of claim 13 further comprising:
a catheter connected to the primary fluid delivery apparatus;
a pressure sensor configured to sense a pressure in a lumen of the catheter; and
the processor configured to analyze the sensed pressure in the lumen of the catheter to identify one or more catheter malfunctions.

21. The system of claim 20, wherein the processor is configured to switch delivery of the vasodilator from the primary fluid delivery apparatus to the reserve fluid delivery apparatus when the analysis of the sensed pressure in the lumen of the catheter identifies one or more catheter malfunctions.

22. The system of claim 13 further comprising at least one fluid delivery device that comprises at least one of the primary fluid delivery apparatus or the reserve fluid delivery apparatus and the processor.

23. The system of claim 13 further comprising a programmer that comprises the processor, wherein the programmer is configured to program at least one of the primary fluid delivery apparatus or the reserve fluid delivery apparatus.

24. The system of claim 13, wherein the sensor comprises at least one of a pressure or an optical blood oxygen saturation sensor.

25. A method comprising:
delivering a vasodilator with a fluid delivery device;
sensing at least one of blood pressure or blood flow in one of a ventricle or an atria of a heart, a pulmonary artery, and a renal vessel with a sensor; and
triggering a therapeutic action by the fluid delivery device when the sensed at least one of blood pressure or blood flow traverses the threshold.

26. The method of claim 25, wherein triggering a therapeutic action comprises triggering an alarm when the sensed at least one of blood pressure or blood flow traverses the threshold.

27. The method of claim 26, wherein the alarm comprises one of an audible, tactile, or visual alert.

28. The method of claim 26, wherein triggering a therapeutic action comprises causing the fluid delivery device to vibrate when the sensed at least one of blood pressure or blood flow traverses the threshold.

29. The method of claim 25, wherein triggering a therapeutic action comprises modifying at least one of a rate, duration, or frequency of delivery of the vasodilator or an amount of the vasodilator delivered by the fluid delivery device when the sensed at least one of blood pressure or blood flow traverses the threshold.

30. The method of claim 25, wherein triggering a therapeutic action comprises controlling the fluid delivery device to switch delivery of the vasodilator from a primary delivery apparatus to a reserve fluid delivery apparatus when the sensed at least one of blood pressure or blood flow traverses the threshold.

31. The method of claim 25 further comprising:
sensing a pressure in a lumen of a catheter connected to the fluid delivery device using a pressure sensor; and
analyzing the pressure in the lumen of the catheter to identify one or more catheter malfunctions.

32. The method of claim 31, wherein the therapeutic action is triggered by the fluid delivery device when the analysis of the pressure in the lumen of the catheter identifies one or more catheter malfunctions.

33. A fluid delivery system comprising:
means for delivering a vasodilator;
means for sensing at least one of blood pressure or blood flow in one of a ventricle or an atria of a heart, a pulmonary artery, and a renal vessel; and
means for triggering a therapeutic action when the sensed at least one of the sensed blood pressure or blood flow traverses the threshold.

34. The system of claim 33 further comprising:
primary means for receiving and storing a first amount of the vasodilator; and
reserve means for receiving and storing a second amount of the vasodilator,
wherein the means for delivering a vasodilator is configured to deliver the vasodilator via one or both of the primary means and the reserve means.
